(19) United States
(12) Patent Application Publication
(10) Pub. No.: US 2023/0077745 A1
(43) Pub. Date: Mar. 16, 2023

(54) RECTUM EXPANDING BALLOON CATHETER

(71) Applicants: THE ASAN FOUNDATION, Seoul (KR); UNIVERSITY OF ULSAN FOUNDATION FOR INDUSTRY COOPERATION, Ulsan (KR)

(72) Inventors: Young Seok KIM, Seoul (KR); In Ha YUN, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 18/056,766

(22) Filed: Nov. 18, 2022

Related U.S. Application Data
(63) Continuation of application No. PCT/KR2021/006108, filed on May 14, 2021.

(30) Foreign Application Priority Data
May 20, 2020 (KR) 10-2020-0060169

(51) Int. Cl. A61M 3/02

(52) U.S. Cl. CPC A61M 3/0295; A61M 2210/1067; A61M 2205/0216

(57) ABSTRACT

Disclosed is a rectum expanding balloon catheter. The rectum expanding balloon catheter includes a catheter body having a rectum insertion part inserted into a rectum and a gripping part extending from the rectum insertion part in a lengthwise direction thereof and that is used to insert the rectum insertion part into the rectum, and in which a fluid flow passage, along which a fluid flows, is formed in an interior thereof, a balloon that expands and contracts an area on an outside of the rectum insertion part and that locates the rectum insertion part in the rectum, and a stopper provided in the catheter body, and that restricts a length, by which the catheter body is inserted into the rectum, while being adhered to a periphery of an anal passage.

### CROSS-REFERENCE TO RELATED APPLICATIONS

The present application is a continuation of International Patent Application No. PCT/KR2021/006108, filed on May 14, 2021, which is based upon and claims the benefit of priority to Korean Patent Application No. 10-2020-0060169 filed on May 20, 2020. The disclosures of the above-listed applications are hereby incorporated by reference herein in their entirety.

### BACKGROUND

Embodiments of the inventive concept described herein relate to a rectum expanding balloon catheter, and more particularly, to a rectum expanding balloon catheter that is inserted into a rectum to fix motion of an affected area.

During a radiation treatment of a prostate gland, a rectum expanding balloon catheter is used to alleviate an influence on a rectum by radiations.

The balloon catheter includes a balloon, and a catheter body that supports the balloon.

Accordingly, according to the balloon catheter, a prostate gland is fixed by inserting a balloon into a rectum, expanding the balloon by using water or air, and pressing a wall of the rectum to the prostate gland such that the wall is adhered to the prostate gland.

By fixing the prostate cancer during the radiation treatment using the balloon catheter, exposure of the parts, except for the prostate cancer, to the radiations may be minimized and a treatment effect may be increased.

Meanwhile, because the catheter body for supporting the balloon has a high strength, a patient may be severely surprised or injured while the catheter body is inserted into the rectum, and because the catheter body cannot be reused, it is difficult to fix the balloon at the same location.

Furthermore, conventionally, when the balloon catheter is inserted into the rectum, a location of the balloon applied to a treatment varies according to tension of sphincteral muscles of an anal passage, whereby reproducibility and a precision of the radiation treatment by using the balloon catheter deteriorate.

### SUMMARY

Embodiments of the inventive concept provide a rectum expanding balloon catheter, by which a length of a catheter body inserted into a rectum is restricted, the catheter body is prevented from being easily moved in the rectum whereby a rectum insertion part of the catheter body is stably located in the rectum and reproducibility and a precision of the radiation treatment may be secured.

Embodiments of the inventive concept also provide a rectum expanding balloon catheter, by which a catheter body may be inserted into a rectum while an interval between a balloon and a stopper may vary in correspondence to various locations of an affected area.

According to an embodiment, a rectum expanding balloon catheter includes a catheter body having a rectum insertion part inserted into a rectum and a gripping part extending from the rectum insertion part in a lengthwise direction thereof and that is used to insert the rectum insertion part into the rectum, and in which a fluid flow passage, along which a fluid flows, is formed in an interior thereof, a balloon that expands and contracts an area on an outside of the rectum insertion part and that locates the rectum insertion part in the rectum, and a stopper provided in the catheter body, and that restricts a length, by which the catheter body is inserted into the rectum, while being adhered to a periphery of an anal passage.

Here, the rectum expanding balloon catheter may further include an anal passage cap provided on one side of the stopper, which faces the rectum, and being attached and detached to and from the anal passage.

Further, the stopper and the anal passage cap may be coupled to the catheter body to be slid while generating sliding frictional forces.

The rectum expanding balloon catheter may further include a plurality of gradations provided in the gripping part at a specific interval along the lengthwise direction of the catheter body, and that indicates the length, by which the catheter body is inserted into the rectum.

The balloon may have different expansion coefficients such that an area of the balloon is expanded to be flat along the lengthwise direction of the catheter body in correspondence to irradiation of radiations and the remaining areas thereof are expanded to have a circular cross-sectional shape.

The balloon may include a rubber or silicon material that is expanded and contracted.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a view illustrating a configuration of a rectum expanding balloon catheter according to an embodiment of the inventive concept;

FIG. 2 is a view illustrating a state, in which the rectum expanding balloon catheter of FIG. 1 is inserted into a rectum; and

FIG. 3 is a view illustrating a state, in which a balloon of the rectum expanding balloon catheter of FIG. 1 is expanded in a rectum.

### DETAILED DESCRIPTION

The above and other aspects, features, and advantages of the inventive concept will become apparent from the following description of the following embodiments given in conjunction with the accompanying drawings. However, the inventive concept is not limited by the embodiments disclosed herein but will be realized in various different forms, and the embodiments are provided only to make the disclosure of the inventive concept complete and fully inform the scope of the inventive concept to an ordinary person in the art, to which the inventive concept pertains.

The terms used herein are provided to describe the embodiments but not to limit the inventive concept. In the specification, the singular forms include plural forms unless particularly mentioned. The terms “comprises” and/or “comprising” used herein does not exclude presence or addition of one or more other components, in addition to the aforementioned components. Throughout the specification, the same reference numerals denote the same components, and “and/or” includes the respective components and all combinations of the components.

Unless otherwise defined, all terms (including technical and scientific terms) used herein have the same meaning as commonly understood by those skilled in the art to which the inventive concept pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

Hereinafter, the inventive concept will be described in detail with reference to the accompanying drawings.

Prior to the description, it should be noted in advance that it will be described that a rectum expanding balloon catheter according to an embodiment of the inventive concept is inserted to treat a prostate gland, but the inventive concept is not limited thereto and the rectum expanding balloon catheter according to the embodiment of the inventive concept may be inserted into the rectum in correspondence to various affected areas.

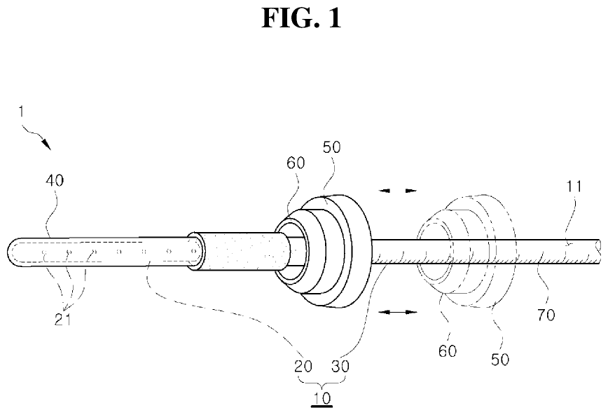

FIG. 1 illustrates a configuration of a rectum expanding balloon catheter according to an embodiment of the inventive concept.

As illustrated in the drawing, a rectum expanding balloon catheter 1 according to an embodiment of the inventive concept includes a catheter body 10, a balloon 40, and a stopper 50.

The catheter body 10 has a hollow pipe shape of a specific length. A fluid flow passage 11, along which a fluid, such as physiological saline or air flows, is formed in an interior of the catheter body 10.

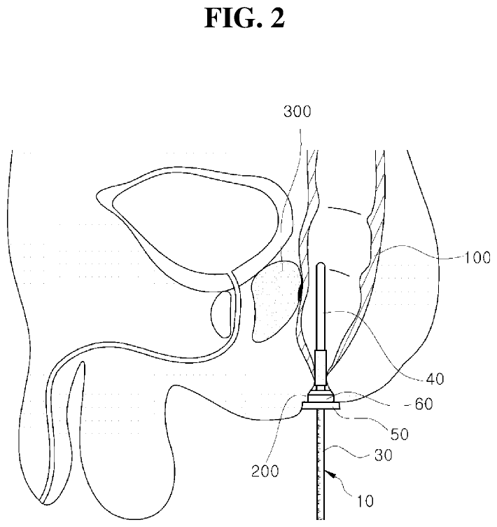

The catheter body 10 includes a rectum insertion part 20 that is inserted into a rectum 100 (see FIG. 2), and a gripping part 30 that extends from the rectum insertion part 20 in a lengthwise direction thereof and is used to insert the rectum insertion part 20 into the rectum 100.

A fluid passage hole 21 for injecting or discharging the fluid to or from the balloon 40 is formed in the rectum insertion part 20. The fluid passage hole 21 is communicated with the fluid flow passage 11 of the catheter body 10.

A tip end of the rectum insertion part 20 may have a streamlined shape such that the catheter body 10 is easily inserted into the rectum 100.

The gripping part 30 is configured to be exposed to an outside of the body of a patient. Furthermore, a fluid injection unit (not illustrated) for injecting or discharging the fluid into or from the balloon 40 is connected to a free end of the gripping part 30.

The balloon 40 is provided in an area of an outside of the rectum insertion part 20 to be expanded and contracted. The balloon 40 is expanded in correspondence to a location of a prostate gland 300 (see FIG. 2) that is the affected area in a state, in which the rectum insertion part 20 of the catheter body 10 is inserted into the rectum 100, to stably locate the rectum insertion part 20 of the catheter body 10 in the rectum 100.

The balloon 40 is expanded in the rectum 100 by the fluid supplied through the fluid passage hole 21 via the fluid flow passage 11 of the catheter body 10 whereby the catheter body 10 is fixed such that the rectum insertion part 20 is prevented from being moved in the rectum 100 and thus the prostate gland 300 is also prevented from being moved. Accordingly, in a state, in which the prostate gland 300 is not moved, radiations may be stably precisely irradiated to the affected area of the prostate gland.

Meanwhile, when the balloon 40 is expanded in the rectum 100, the balloon 40 is expanded along a circumference of the catheter body 10.

Then, the balloon 40 may have different expansion coefficients such that an area of the balloon 40 is expanded to be flat along the lengthwise direction of the catheter body 10 in correspondence to irradiation of radiations and the remaining areas thereof are expanded to have a circular cross-sectional shape.

Accordingly, as the area of the balloon 40, which is expanded to be flat, is located to face the prostate gland 300, a precision of the radiation treatment applied to a prostate cancer may be further enhanced.

Here, the balloon 40 is not limited thereto, and as another embodiment, it may be expanded along a circumference of the catheter body 10 to have a spherical shape.

Meanwhile, the balloon 40 may include a rubber or silicon material that may be expanded and contracted.

The stopper 50 has a disk shape and is coupled to the catheter body 10, and is adhered to a periphery of an anal passage 200 (see FIG. 2) to restrict a length, by which the catheter body 10 is inserted into the rectum 100.

In this way, the stopper 50 prevents the catheter body 10 from easily moving in the rectum 100 by a force that is reflexly applied according to tension of sphincteral muscles of the anal passage whereby the rectum insertion part 20 of the catheter body 10 is stably located in the rectum 100 and reproducibility and a precision of the radiation treatment may be secured.

Here, although not illustrated, the stopper may be firmly fixed to the catheter body by fastening or unfastening the stopper to or from the catheter body by using a clamp screw-coupled to the stopper. Accordingly, the catheter body may be prevented from being easily moved in the rectum.

Furthermore, the rectum expanding balloon catheter 1 according to the embodiment of the inventive concept may further include an anal passage cap 60.

The anal passage cap 60 has a circular cross-sectional shape, and is provided to protrude from one side of the stopper 50 that faces the rectum 100 and is attached to or detached from the anal passage 200.

In this way, by using the anal passage cap 60, the sphincteral muscles of the anal passage fastens the anal passage cap 60 when the catheter body 10 is inserted into the rectum 100 and the anal passage cap 60 is mounted in the anal passage 200. Accordingly, the stopper 50 and the anal passage cap 60 are stably adhered to a periphery of the anal passage 200, and thus the catheter body 10 is fixed to the rectum 100.

Meanwhile, the stopper 50 and the anal passage cap 60, as indicated by a dotted line in FIG. 1, may be coupled to be movable along a lengthwise direction of the catheter body 10. The stopper 50 and the anal passage cap 60 preferably may be sliding-coupled to the catheter body 10 while generating sliding frictional forces so as not to be easily moved from the catheter body 10 by an external force.

In this way, because the stopper 50 and the anal passage cap 60 are coupled to the catheter body 10 to be movable, the catheter body 10 may be inserted into the rectum 100 while an interval between the balloon 40 and the stopper 50 may vary in correspondence to various locations of the affected area. Further, as the interval between the balloon 40 and the stopper 50 is set, the balloon 40 may be precisely located in the rectum whenever the catheter body 10 is inserted into the rectum 100 and reproducibility and a precision of the radiation treatment may be secured.

A plurality of gradations 70 are formed in the gripping part 30 of the catheter body 10 of the rectum expanding balloon catheter 1 according to the embodiment of the inventive concept.

The plurality of gradations 70 are provided in the gripping part 30 at a specific interval along the lengthwise direction of the catheter body 10 to indicate the length, by which the gripping part 30 is inserted into the rectum 100.

Accordingly, in correspondence to various locations of the affected area, the catheter body 10 may be inserted into the rectum 100 after the stopper 50 is moved to the corresponding gradation 70 in consideration of an interval between the affected area and the anal passage 200.

By using the configuration, a process of inserting the rectum expanding balloon catheter 1 according to the embodiment of the inventive concept into the rectum 100 to perform a radiation treatment on the prostate gland that is an affected area will be described as follows.

First, after identifying a distance from the anal passage 200 to the prostate gland 300 that is to be treated through a CT or the like, the stopper 50 and the anal passage cap 60 are moved to the corresponding gradation 70 of the catheter body 10 in consideration of the interval between the affected area and the anal passage 200 such that the balloon 40 is located around the affected area.

Next, in a state, in which the balloon 40 is contracted, the rectum insertion part 20 of the catheter body 10 is inserted into the rectum 100 through the anal passage 200. When the rectum insertion part 20 is inserted into the rectum 100 through the anal passage 200, the catheter body 10 is prevented from being further inserted into the rectum 100 by the stopper 50 when the anal passage cap 60 is inserted into the anal passage 200 and the stopper 50 is adhered to a periphery of the anal passage 200 as illustrated in FIG. 2.

Furthermore, when the catheter body 10 is completely inserted into the rectum 100, the fluid is injected into the fluid flow passage 11 of the catheter body 10 through a fluid injection unit.

The fluid that flows along the fluid flow passage 11 of the catheter body 10 is injected into the balloon 40 provided in the rectum insertion part 20 while being contracted via the fluid passage hole 21 of the rectum insertion part 20.

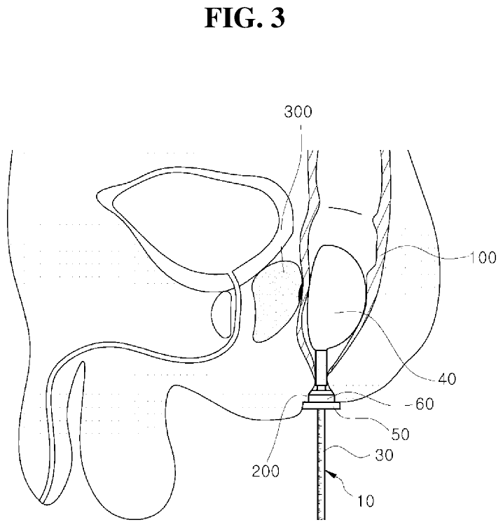

As the fluid is injected into the balloon 40, the balloon 40 is expanded as illustrated in FIG. 3, and locates the rectum insertion part 20 in the rectum 100 while adhering and fixing the rectum insertion part 20 to an interior of the rectum 100. Then, the catheter body 10 is supported in the anal passage 200 and at a periphery of the anal passage 200 by the stopper 50 and the anal passage cap 60 whereby the catheter body 10 is prevented from being easily moved in the rectum 100 by a force that is reflexly applied according to tension of the sphincteral muscles of the anal passage.

Accordingly, the rectum expanding balloon catheter 1 according to the embodiment of the inventive concept is fixed after being inserted into the rectum 100.

Meanwhile, radiations are irradiated toward the prostate gland 300 that is the affected area for treatment in a state, in which the balloon 40 of the rectum expanding balloon catheter 1 is expanded and the catheter body 10 is fixed in the rectum 100.

In this way, according to the inventive concept, the length, by which the catheter body is inserted into the rectum, may be restricted by providing the stopper in the catheter body whereby the catheter body may be prevented from being easily moved in the rectum by a force that is reflexly applied according to tension of the sphincteral muscles of the anal passage and the rectum insertion part of the catheter body may be stably located in the rectum and reproducibility and a precision of the radiation treatment may be secured.

Furthermore, because the stopper is coupled to the catheter body to be movable, the catheter body may be inserted into the rectum while an interval between the balloon and the stopper may vary in correspondence to various locations of the affected area.

Furthermore, because the stopper and the anal passage cap may be sliding-coupled to the catheter body 10 while generating sliding frictional forces whereby the interval between the balloon and the stopper may be set in correspondence to various locations of the affected area such that the catheter body is inserted into the rectum, and the balloon may be precisely located in the rectum whenever the catheter body is inserted into the rectum and reproducibility and a precision of the radiation treatment may be further enhanced.

Although the exemplary embodiments of the inventive concept have been described with reference to the accompanying drawings, it will be understood by those skilled in the art to which the inventive concept pertains that the inventive concept can be carried out in other detailed forms without changing the technical spirits and essential features thereof. Therefore, the above-described embodiments are exemplary in all aspects, and should be construed not to be restrictive.

What is claimed is:

1. A rectum expanding balloon catheter comprising:
a catheter body having a rectum insertion part inserted into a rectum and a gripping part extending from the rectum insertion part in a lengthwise direction thereof and configured to be used to insert the rectum insertion part into the rectum, and in which a fluid flow passage, along which a fluid flows, is formed in an interior thereof;
a balloon configured to expand and contract an area on an outside of the rectum insertion part and configured to locate the rectum insertion part in the rectum; and
a stopper provided in the catheter body, and configured to restrict a length, by which the catheter body is inserted into the rectum, while being adhered to a periphery of an anal passage.

2. The rectum expanding balloon catheter of claim 1, wherein the stopper is coupled to the catheter body to be movable.

3. The rectum expanding balloon catheter of claim 1, further comprising:
an anal passage cap provided on one side of the stopper, which faces the rectum, and being attached and detached to and from the anal passage.

4. The rectum expanding balloon catheter of claim 3, wherein the stopper and the anal passage cap are coupled to the catheter body to be slid while generating sliding frictional forces.

5. The rectum expanding balloon catheter of claim 1, further comprising:
a plurality of gradations provided in the gripping part at a specific interval along the lengthwise direction of the catheter body, and configured to indicate the length, by which the catheter body is inserted into the rectum.

6. The rectum expanding balloon catheter of claim 1, wherein the balloon has different expansion coefficients such that an area of the balloon is expanded to be flat along the lengthwise direction of the catheter body in correspondence to irradiation of radiations and the remaining areas thereof are expanded to have a circular cross-sectional shape.

7. The rectum expanding balloon catheter of claim 1, wherein the balloon includes a rubber or silicon material that is expanded and contracted.
